(19) United States
(12) Patent Application Publication
(10) Pub. No.: US 2005/0002868 A1
(43) Pub. Date: Jan. 6, 2005

(54) Method of treatment of a female suffering from androgen insufficiency

(75) Inventors: Igor Gonda, South Yarra (AU); Nina Frances Wilkins, Port Melbourne (AU); Andrew Jonathan Humberstone, Port Melbourne (AU)

Correspondence Address: FOLEY AND LARDNER;SUITE 500, 3000 K STREET NW, WASHINGTON, DC, 20007, US

(73) Assignee: Acrux DDS Pty Ltd.

(21) Appl. No.: 10/870,546

(22) Filed: Jun. 18, 2004

Related U.S. Application Data
(60) Provisional application No. 60/480,236, filed on Jun. 23, 2003.

(51) Int. Cl. A61L 9/04; A61K 31/57

(52) U.S. Cl. 424/45; 514/177

(57) ABSTRACT

A method of treatment of a female suffering from androgen insufficiency comprising administering to at least one of the abdomen and forearm of the female a transdermal spray or aerosol comprising an androgen.

### FIELD OF THE INVENTION

The present invention relates to a method for the treatment of a female suffering from androgen insufficiency whereby the method the invention provides a method for controlling the extent and/or profile of transdermal release of an androgen, the method including the step of applying a composition containing the physiologically active agent, a drug penetration enhancer and a volatile solvent to an anatomical site of a woman to control the androgen serum concentration profile.

### BACKGROUND OF THE INVENTION

Low serum androgen levels in women are associated with a range of clinical symptoms including loss of libido, lack of general well being and fatigue. A major deterrent to widespread use of androgen therapy at the present time is a lack of preparations suitable for use in women. To date, the only androgen therapy available for women in the United States is an oral preparation which has been associated with suppression of high density lipoproteins after long term administration.

Conventional means for administering therapeutic agents such as androgens to a human or animal are usually limited to some degree by biological, chemical, and physical barriers. Examples of physical barriers are the skin and various organ membranes that must be traversed before the agent reaches a target. Chemical barriers include pH variations, lipid bi-layers, and degrading enzymes. Both biologically and chemically active agents are particularly vulnerable to such barriers.

Several studies have shown a beneficial effect of transdermally administered androgens, in particular testosterone, on the symptoms associated with low serum androgen levels in women (Braunstein et al., 2002, Fertility and Stability, 77(4), S94-99).

Transdermal delivery of therapeutic agents offers several inherent clinical and patient advantages over traditional oral tablet and capsule formulations, especially for drugs that:
cannot safely be given orally, for example because of irritant effects on the gastrointestinal tract
undergo extensive so-called ‘first-pass’ metabolism and are thus substantially inactivated in the liver immediately after oral administration
are poorly absorbed or poorly bioavailable after oral administration

Administration of therapeutic agents through the skin (‘transdermal drug delivery’) has received increased attention because it not only provides a relatively simple dosage regime but it also provides a relatively slow and controlled route for release of an agent into the systemic circulation. However, transdermal drug delivery is complicated by the fact that the skin behaves as a natural barrier and therefore transport of agents through the skin is a complex mechanism.

Structurally, the skin consists of two principle parts, a relatively thin outermost layer (the ‘epidermis’) and a thicker inner region (the ‘dermis’). The outermost layer of the epidermis (the ‘stratum corneum’) consists of flattened dead cells which are filled with keratin. The region between the flattened dead cells of the stratum corneum is filled with lipids which form lamellar phases that are responsible for the natural barrier properties of the skin. Epidermal thickness is remarkably constant over the body, except on the soles of the feet and the palms of the hand (Rushmer, et al., 1966, The Skin. Science 154(3747), 343-348).

For effective transdermal delivery of a therapeutic agent that is applied to the surface of the skin (‘topical application’), the agent must be partitioned firstly from the vehicle into the stratum corneum, it must typically then be diffused within the stratum corneum before being partitioned from the stratum corneum to the viable epidermis and dermis and then into the bloodstream.

To overcome some of the problems with transdermal delivery that are associated with transport across the dermal layers (‘percutaneous absorption’), physiologically active agents can be formulated with incorporation of one or more drug penetration enhancers. For example, aqueous ethanol can be used as a vehicle in formulations for topical application. Ethanol can act as a penetration enhancer that can increase the flux of an active agent across the skin due to a solvent drag effect (Berner et al., 1989, J. Pharm. Sci, 78(5), 402-406). Octyl para-methoxycinnamate (Padimate O), Octyl salicylate and Azone™ are further examples of penetration enhancers that have been shown to improve percutaneous absorption (see U.S. Pat. No. 6,299,900).

There is a need for improved compositions for transdermal delivery of androgens to a female suffering from androgen insufficiency.

No admission is made that any reference, including any patent or patent document, cited in this specification constitutes prior art. In particular, it will be understood that, unless otherwise stated, reference to any document herein does not constitute an admission that any of these documents forms part of the common general knowledge in the art in Australia or in any other country. The discussion of the references states what their authors assert, and the applicant reserves the right to challenge the accuracy and pertinency of any of the documents cited herein.

### SUMMARY OF THE INVENTION

The present invention arises from the inventor's studies of finite dose formulations which contain penetration enhancers that enhance the percutaneous absorption of an androgen relative to the anatomical site of application. The inventor's studies have shown that the extent and/or profile of transdermal release of an androgen may be modified to produce either a substantially zero order steady state or a diurnal (morning peak) serum testosterone profile, as desired.

The present invention provides a method of treatment of a female suffering from androgen insufficiency comprising administering to at least one of the abdomen and forearm of the female a transdermal spray comprising an androgen.

The present invention in a preferred embodiment provides a method for controlling the free androgen serum concentration within the bloodstream of a female suffering from androgen insufficiency, the method including the step of delivering a transdermal spray comprising an androgen to either:
(a) the abdomen of the female to provide a substantially zero order steady state free androgen blood serum profile; and/or
(b) the forearm of the female to provide a diurnal peak free androgen blood serum profile,
   wherein administration of the androgen to the abdomen and/or the forearm is used to control the free androgen serum concentration within the bloodstream of the female.

According to the method of the invention the androgen may be delivered to the abdomen to achieve a substantially zero order steady state free androgen blood serum profile in the female suffering from androgen insufficiency. The blood serum profile of the androgen in the systemic circulation preferably approaches zero order in nature so as to reduce the ratio of maximum concentration (Cₘₐₓ to C_{avg}) for the androgen over the dosage interval. For example a Cₘₐₓ to C_{avg} ratio is preferably less than 1.5 after the subject has applied the daily dose of the composition for at least 5 consecutive days.

Also according to the method of the invention the androgen may be delivered to the forearm to achieve a diurnal peak free androgen blood serum profile in the female suffering from androgen insufficiency. In this way it is possible to increase the initial burst of androgen across the skin before a plateau in the blood serum profile. By modulating diurnal variation, the free testosterone serum concentration may be maintained with a ratio of the Cₘₐₓ to C_{avg} greater than 1.5 after the subject has applied the daily dose of the composition for at least 5 consecutive days.

Administration to the forearm and abdomen may be used concomitantly to provide, for example, a modified release of androgen by delivering the androgen to the forearm, followed by a stable free androgen serum concentration by delivering the androgen to the abdomen of the female.

The present invention also provides a method for the treatment of a woman suffering from androgen insufficiency, the method including the step of administering a transdermal spray containing an androgen to either:
(a) the abdomen of the female to provide a substantially zero order steady state free androgen blood serum profile; and/or
(b) the forearm of the female to provide a diurnal peak free androgen blood serum profile,
   wherein administration of the androgen to the abdomen and/or the forearm is used to control the free androgen serum concentration within the bloodstream of the female.

The present invention also provides a method of enhancing percutaneous absorption of an androgen, the method including the step of applying a composition containing the androgen, a drug penetration enhancer and a volatile solvent to the skin of a host to form an amorphous deposit of the androgen and the penetration enhancer upon evaporation of the volatile solvent such that partitioning of the androgen from the stratum corneum to the viable epidermis is enhanced.

The androgen is preferably administered at a dose rate of 20 to 400 micrograms per day by applying the composition of the invention using an amount of androgen ranging from 10 to 300 micrograms per square centimetre, applied over a surface area of 10 to 75 square centimetres, typically once a day.

In a further embodiment the invention provides a metered dose spray applicator containing a composition that includes an androgen, a drug penetration enhancer and a volatile solvent wherein the applicator can be used for administration of the androgen to the skin of a host.

### BRIEF DESCRIPTION OF THE FIGURES

In the accompanying figures:

FIG. 1 is a graph showing the mean (±SEM) serum concentrations profiles on day 5 for free testosterone after application of a 2×91 μl sprays from testosterone composition for 5 days to the abdomen or forearm.

### DETAILED DESCRIPTION OF THE INVENTION

Before describing the present invention in detail, it is to be understood that this invention is not limited to specific drug delivery systems, device structures, enhancers or carriers, as such may vary. It is also to be understood that the terminology used is for the purpose of describing particular embodiments only, and is not intended to be limiting.

In describing the present invention, the following terminology will be used in accordance with the definitions set out below.

The term “androgen” as used herein refers to any male hormone that is responsible for changes in body shape (muscle gain, fat distribution) and male secondary sexual characteristics.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. The present method of “treating” a patient, as the term is used herein, thus encompasses both prevention of a disorder in a predisposed individual and treatment of the disorder in a clinically symptomatic individual.

By “transdermal” drug delivery is meant administration of a drug to the skin surface of an individual so that the drug passes through the skin tissue and into the individual's blood stream, thereby providing a systemic effect.

The term “stable free androgen serum concentration” as used herein refers to a free androgen serum concentration with a ratio of the Cₘₐₓ to C_{avg} less than 1.5 after the subject has applied the daily dose of the composition for at least 5 consecutive days.

As mentioned previously, the present invention provides a method suitable for providing a free androgen serum concentration within the bloodstream of a female. The method includes the step of administering a transdermal spray containing an androgen to the abdomen or forearm of the female. In addition the invention provides a method for the treatment of a female suffering from androgen insufficiency. The method includes the step of administering a transdermal spray containing an androgen to the abdomen or forearm of the female such that a controlled free androgen serum concentration is obtained.

A benefit of the method of the present invention is that the androgen blood serum level may be controlled, which means that provided the circulating androgen concentrations are kept within, or close to, the upper limit of the normal physiological range, masculinizing effects are extremely unlikely. In contrast with other transdermal preparations, the diurnal variation in serum levels may be modified or eliminated thereby reducing side effects. Thus according to the method of the present invention an androgen can be administered continuously without encountering the skin irritation problems of occlusive transdermal patches such as those described in U.S. Pat. No. 5,460,820.

In a preferred form of the invention the androgen is administered to the abdomen. We have surprisingly found that a stable free testosterone serum concentration within the bloodstream can be achieved by application of the spray to the stomach. In contrast to other parts of the body which could be used for transdermal administration, we have found the abdomen to provide a surprisingly zero order stable serum level.

In another preferred form of the invention the androgen is administered the forearm. We have surprisingly found that a diurnal profile of free testosterone serum concentration with a morning peak profile within the bloodstream can be achieved by application of the spray to the forearm. In contrast to other parts of the body which could be used for transdermal administration, we have found the forearm to provide a surprisingly consistent diurnal profile that is exemplified by a morning peak of free testosterone within the bloodstream.

A combination of administration to the abdomen and the forearm may provide further means for modifying and controlling the free androgen serum concentration in the bloodstream.

Preferred physiologically acceptable androgens include testosterone, testosterone propionate, testosterone enanthate, testosterone cypionate, methyltestosterone, dihydrotestosterone (DHT), dehydroepiandrostenedione (DHEA), fluoxymesterone, danazol, calusterone, dromostanolone propionate, ethylestrenol, methandriol, methandrostenolone, nandrolone decanoate, nandrolone phenpropionate, oxandrolone, oxymetholone, stanozolol, MENT (7-methyl-19-testosterone) and testolactone or a pharmaceutically acceptable salt or derivative of any one of the aforementioned. More preferably the physiologically acceptable agent is testosterone.

The amount of physiologically acceptable androgen present in the composition of the invention is preferably in the range of 0.1 to 10% by weight and more preferably 2 to 8% by weight.

The composition of the invention also includes a penetration enhancer. The preferred penetration enhancers for use in the composition of the invention are sunscreen esters, such as those selected from the group consisting of C₈ to C₁₈ alkylcinnamate, C₈ to C₁₈ alkylmethoxycinnamate, C₈ to C₁₈ alkyl salicylate and mixtures thereof. More preferably the penetration enhancers are selected from padimate O and octyl salicylate.

The amount of penetration enhancer present in the composition of the invention is preferably in the range of 0.1 to 10% w/v and more preferably 2 to 8% w/v.

The composition of the invention preferably also contains a volatile solvent. Preferably the volatile solvent has a vapour pressure is above 35 mm Hg at atmospheric pressure and normal skin temperature of 32° C. In a particularly preferred form of the invention the volatile solvent is a lower alcohol, more preferably ethanol or isopropanol, or a mixture thereof. Typically the solvent will be present in an amount of from 40 to 80% v/v and more preferably 50 to 70% v/v.

The spray may be in the form of a mist fog or the like and may be delivered as an aerosol with a pressurised gaseous dispersion medium. Alternatively the spray may be delivered by a pump pack or other similar spray device of the type known in the industry.

Conveniently, the composition is a topical spray composition that contains the androgen, the drug penetration enhancer and the volatile solvent and the method includes the step of spraying the composition onto the abdomen or forearm of a female to achieve a controlled androgen serum concentration within the bloodstream.

The amount of androgen administered will depend on a number of factors and will vary from subject to subject and depend on the particular androgen administered, the severity of the symptoms, the subject's age, weight and general condition, and the judgment of the prescribing physician. The minimum amount of androgen is determined by the requirement that sufficient quantities of the androgen must be present in the composition to maintain the desired rate of release over the given period of application. The maximum amount for safety purposes is determined by the requirement that the quantity of drug present cannot exceed a rate of release that reaches toxic levels. Generally, the maximum concentration is determined by the amount of agent that can be received without producing adverse histological effects such as irritation, an unacceptably high initial pulse of agent into the body. Of course it will be appreciated by those skilled in the art that the desired dose of a specific androgen will depend on the nature of the androgen as well as on other factors; the minimum effective dose of each androgen is of course preferred.

In the case of testosterone and application to the abdomen;
The stable free testosterone serum concentration is preferably maintained between about 4 pg/ml and about 8 pg/ml after the subject has applied the daily dose of the composition for at least 5 consecutive days;
Preferably, the stable free testosterone serum concentration is substantially maintained with a ratio of the Cₘₐₓ to C_{avg} less than 1.5 after the subject has applied the daily dose of the composition for at least 5 consecutive days;
The preferred method of androgen serum analysis is equilibrium dialysis, whereby the percent free androgen is calculated from the ratio of radioactivity outside the cell versus inside the cell, multiplied by the total testosterone in the serum (corrected for units) thus giving a concentration of free testosterone in the serum (Esoterix Endocrinology Inc.);
The degree of fluctuation (Cₘₐₓ−Cₘᵢₙ)/C_{avg}×100%) of the present invention is preferably maintained below 80%;
The stable free testosterone serum concentration is preferably maintained between 50 and 100% of the normal range for women whereby the normal range is considered to be between 1.1 and 6.3 pg/ml.

In the case of testosterone and application to the forearm;
The free testosterone serum concentration is preferably maintained between about 4 pg/ml and about 20 pg/ml after the subject has applied the daily dose of the composition for at least 5 consecutive days;
Preferably, the stable free testosterone serum concentration is substantially maintained with a ratio of the Cₘₐₓ to C_{avg} greater than 1.5 after the subject has applied the daily dose of the composition for at least 5 consecutive days;
The preferred method of androgen serum analysis is equilibrium dialysis, whereby the percent free androgen is calculated from the ratio of radioactivity outside the cell versus inside the cell, multiplied by the total testosterone in the serum (corrected for units) thus giving a concentration of free testosterone in the serum (Esoterix Endocrinology Inc.);
The time to maximum concentration (Tₘₐₓ) is greater than 15 hours;
The degree of fluctuation (Cₘₐₓ−Cₘᵢₙ)/C_{avg}×100%) of the present invention is preferably maintained above 100%.

A preferred composition of the present invention may contain from about 0.1% to about 10% of an androgen, from about 0.1% to about 10% of the dermal penetration enhancer, and from about 85% to about 99.8% of the volatile solvent by weight.

Optionally, the composition may have additional pharmaceutical excipients, for example gelling agents, such as carbopol and cellulose derivatives.

The composition may also include a hydrofluorocarbon propellant, such as HFC-134a, which may together with the volatile pharmaceutically acceptable solvent form a single-phase carrier solution of the active agent. In this form of the invention the transdermal spray may be in the form of an aerosol.

The invention will now be described with reference to the following examples. It is to be understood that the examples are provided by way of illustration of the invention and that they are in no way limiting to the scope of the invention.

### EXAMPLE 1

Method

The study was a single centre, open label pharmacokinetic study, with a randomised, two-way, cross-over design. The two treatment periods were 5 days with an eight day washout between treatments. Intensive blood sampling was performed on day 5 of each treatment period for pharmacokinetic analysis. The treatments consisted of daily application of 2×91 μL sprays for 5 days, either applied to the abdomen or forearm. Free serum testosterone concentration profiles were measured over 24 hours after daily administration of transdermal testosterone for 5 days to 6 healthy surgically menopausal women stabilized on oral oestrogen therapy. HPLC separation (followed by RIA) and equilibrium dialysis were used to measure free testosterone (Esoterix Inc.).

Composition

| | |
|---|---|
| Testosterone | 5% w/v |
| Octyl salicylate | 8% w/v |
| Ethanol (95%) | to volume |

Result

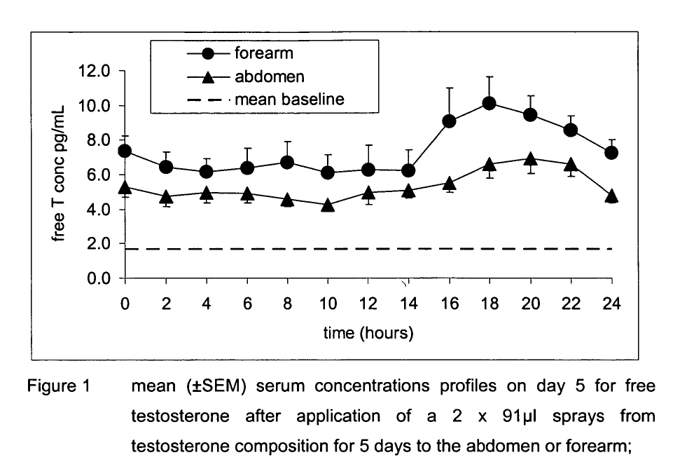

Steady-state concentrations of free testosterone were attained on day 5. Although average serum concentrations of free testosterone were significantly higher after application to the forearm, there was considerably less variation after dose application to the abdomen, as shown in FIG. 1.

Once a day application of the preferred composition to the abdomen was confirmed as the dose which elevated average free testosterone levels of postmenopausal women with low serum testosterone levels into the mid-to-high normal range for premenopausal women, with C_{avg} levels maintained at 5.3 (±1.9), as shown in table 1.

TABLE 1
Mean (±s.d.) pharmacokinetic parameters for free T after
application of 2 × 91 μL sprays from the preferred
composition for 5 days to the abdomen or forearm

| | abdomen | forearm |
|---|---|---|
| Baseline (pg/mL) | 1.7 ± 0.6 | 1.7 ± 0.5 |
| Cavg (pg/mL) | 5.3 ± 1.9 | 7.4 ± 3.3* |
| Cmax (pg/mL) | 7.8 ± 3.2 | 13 ± 8* |
| Cmin (pg/mL) | 3.7 ± 1.3 | 4.4 ± 2.5 |
| Tmax (hours) | 14 ± 7 | 18 ± 5 |
| DF (%) | 74 ± 26 | 109 ± 50 |

The normal range for premenopausal women is 1.1-6.3 pg/mL
*Significantly different to abdomen (p < 0.05)

Finally, it is understood that various other modifications and/or alterations may be made without departing from the spirit of the present invention as outlined herein.

1. A method of treatment of a female suffering from androgen insufficiency comprising administering to at least one of the abdomen and forearm of the female a transdermal spray comprising an androgen.

2. A method according to claim 1 wherein the spray is applied to the abdomen of the female so as to provide a zero order serum concentration of the androgen within the bloodstream.

3. A method according to claim 2 wherein the stable free androgen serum concentration is substantially maintained between about 4 pg/ml serum to about 8 pg/ml serum after the subject has applied the daily dose of the composition for at least 5 consecutive days.

4. A method according to claim 2 wherein the stable free androgen serum concentration is substantially maintained with a ratio of the Cₘₐₓ to C_{avg} less than 1.5.

5. A method according to claim 2 wherein the degree of fluctuation is maintained below 80%.

6. A method according to claim 2 wherein the stable free androgen serum concentration is maintained between 50 and 100% of the normal range for women.

7. A method according to claim 1 wherein an amount of androgen is applied to the forearm of the female so as to achieve a diurnal peak in the free androgen serum concentration within the bloodstream.

8. A method according to claim 7 wherein the free androgen serum concentration is substantially maintained between about 4 pg/ml serum to about 20 pg/ml serum after the subject has applied the daily dose of the composition for at least 5 consecutive days.

9. A method according to claim 7 wherein the diurnal peak in free androgen serum concentration is substantially maintained with a ratio of the Cₘₐₓ to C_{avg} greater than 1.5.

10. A method according to claim 7 wherein the degree of fluctuation is maintained above 100%.

11. A method according to claim 1 wherein administration to the forearm and abdomen is used concomitantly to provide a modified release of androgen by delivering the androgen to the forearm, followed by a stable free androgen serum concentration by delivering the androgen to the abdomen of the female.

12. A method according to claim 1 wherein the androgen is selected from the group consisting of testosterone propionate, testosterone enanthate, testosterone cypionate, methyltestosterone, dihydrotestosterone (DHT), dehydroepiandrostenedione (DHEA), fluoxymesterone, danazol, calusterone, dromostanolone propionate, ethylestrenol, methandriol, methandrostenolone, nandrolone decanoate, nandrolone phenpropionate, oxandrolone, oxymetholone, stanozolol, MENT (7-methyl-19-testosterone) and testolactone or a pharmaceutically acceptable salt or derivative of any one of the aforementioned.

13. A method according to claim 1 wherein the androgen is testosterone.

14. A method according to claim 1 wherein the transdermal spray comprises:
a) a therapeutically effective amount of an androgen
b) at least one dermal penetration enhancer.

15. A method according to claim 14 wherein the spray comprises on a weight basis:
a) from about 0.1 to 10% of said androgen
b) from about 0.1 to 10% of said at least one penetration enhancer.

16. A method according to claim 14 wherein the spray further comprises at least one volatile solvent, wherein the volatile solvent has a vapour pressure above 35 mmHg at atmospheric pressure and a temperature of 32° C.

17. A method according to claim 16, wherein at least one volatile solvent is selected from ethanol and isopropanol or a mixture thereof.

18. A method according to claim 14 wherein at least one dermal penetration enhancer is a lipophilic liquid having a vapour pressure below 10 mmHg at atmospheric pressure and a temperature of 32° C. and a molecular weight in the range of from 200 go 400 Daltons.

19. A method according to claim 14 wherein at least one dermal penetration enhancers is selected from the group consisting of oleic acid, oleyl alcohol, cyclopentadecanone (CPE-218™), sorbitan monooleate, glycerol monooleate, propylene glycol monolaurate, polyethylene glycol monolaurate, 2-n-nonyl 1,3-dioxolane (SEPA™), dodecyl 2-(N,N-dimethylamino)-propionate (DDAIP) or its salt derivatives, 2-ethylhexyl 2-ethylhexanoate, isopropyl myristate, dimethyl isosorbide, 4-decyloxazolidinon-2-one (SR-38™, TCPI, Inc.), 3-methyl-4-decyloxazolidinon-2-one, octyl dimethyl-para-aminobenzoate, octyl para-methoxycinnamate, octyl salicylate and mixtures thereof.

20. A method according to claim 14 wherein at least one dermal penetration enhancer is selected from safe skin-tolerant ester sunscreens.

21. A method according to claim 14 wherein the composition comprises at least one additional component selected from the group consisting of active agents, co-solvents, surfactants, emulsifiers, antioxidants, preservatives, stabilisers, diluents and mixtures of two or more of said components.
